(19) United States
(12) Patent Application Publication
(10) Pub. No.: US 2006/0024229 A1
(43) Pub. Date: Feb. 2, 2006

(54) Method and product for locating an internal bleeding site

(76) Inventors: Seth Karp, Boston, MA (US); Evan Garfein, Boston, MA (US)

Correspondence Address: FRISHAUF, HOLTZ, GOODMAN & CHICK, PC, 220 5TH AVE FL 16, NEW YORK, NY, 10001-7708, US

(21) Appl. No.: 10/903,303

(22) Filed: Jul. 29, 2004

(51) Int. Cl. A61K 51/00; A61K 38/48; A61K 38/36; A61K 35/14

(52) U.S. Cl. 424/1.11; 514/12; 424/94.64

(57) ABSTRACT

A method is provided for localizing an internal bleeding site whereby a protein or other factor involved in the clotting process is complexed to an imaging agent and injected into a patient believed to be at risk of internal bleeding. A clot in the patient will naturally accumulate a certain concentration of the injected complex, and within a short period of time the concentration becomes sufficient to be detected by an imaging apparatus. The imaging contrast agent may, for example, be an MRI contrast agent, a CT contrast agent, a PET agent, or a fluorescent substance.

### FIELD OF THE INVENTION

This invention relates to medical diagnostic techniques and more particularly to a technique for locating an internal bleeding site in a human body.

### BACKGROUND OF THE INVENTION

Bleeding is a common reason for hospitalization and outpatient treatment, particularly in older individuals. Disease or trauma can cause a hemorrhage at virtually any location in the body. Bleeding through the skin is easily identified, since it is clearly visible. However, bleeding within an internal cavity or organ of the body can be much more difficult to identity. As such, critical treatment may be delayed as time is spent attempting to localize the internal hemorrhage.

Internal bleeding is an important health problem. Approximately 1 in 5 people will experience at least one episode of significant internal bleeding during their lifetime. About half of these episodes are due to bleeding from the colon. The most common cause of colonic bleeding is diverticulosis. Approximately sixty-five percent (65%) of people develop this condition by age eighty-five. Fifteen percent (15%) of these people, or approximately ten percent (10%) of the entire population will experience significant bleeding as a result.

Many diagnostic techniques now exist to localize an area of internal hemorrhage. These techniques include endoscopy, angiography and nuclear medicine scans.

Endoscopy involves the placing of an optical scope into a body orifice such as the esophagus stomach or large bowel. Once a bleeding site is visualized, treatment is often possible using well-known techniques such as cauterization or banding. In general, this technique requires that active bleeding occur during the viewing procedure. Bleeding sites may be difficult to identify because of obscuration by blood and the fact that certain regions of the bowel and most internal organs are inaccessible to scopes.

Angiography is an invasive procedure involving the passage of a catheter into the patient's aorta through an entry site, usually in the leg. Dye is injected from the end of the catheter when the catheter is located adjacent to vessels in which bleeding is likely occurring. The dye pools in an area of active bleeding, producing a characteristic blush that can be seen using an X-ray camera. There are many disadvantages of this technique. The dye can cause reactions within the body resulting in kidney failure or even death. Serious bleeding can occur at the site through which the catheter is inserted, and at times this requires an operation to repair. In addition, high doses of X-rays are required to perform this test.

Finally, a nuclear medicine scan involves the injection, into the patient's blood stream, of a radioactive marker that attaches to red blood cells. The cells are traced to an active bleeding site using remote scanners sensitive to radiation.

Each of the diagnostic tests described above requires active bleeding to reveal the presence of an internal hemorrhage. It is often the case, however, that by the time the diagnostic tests are performed, a clot produced by the body has stopped the bleeding, so the test does not provide useful information. Without definitive treatment of the bleeding source about half of patients will experience a recurrence of the hemorrhage. This is a dangerous situation because the bleeding may occur at any time, even after the patient has left the hospital.

The ability to localize a bleeding source, even when no active bleeding is occurring, would allow definitive treatment in many cases and greatly reduce the potential harm caused by bleeding. In cases of particularly severe hemorrhage, the inability to accurately localize a bleeding site may mean that the surgery required to correct it ends up being much larger in scale than would be required if the bleeding source had been clearly identified. The disadvantages to a larger operation are clear. It increases time and costs, increases complications and requires a longer recovery time. For example, if a patient has a life-threatening bleeding condition in the colon, inability to accurately define the region necessitates removal of the entire colon. Conversely, if localization were possible, the patient might lose only one quarter of the entire colon. The latter operation is shorter, simpler and the patient suffers no substantial disability following the operation.

Accordingly, it is an object of this invention to provide a method for localizing an internal bleeding site/hemorrhage that does not require substantial and invasive procedures or internal visible observations. This invention operates by recognizing a consequence of the bleeding—i.e., a clot formed by the body at the site of bleeding. This clot is produced during active bleeding and persists after the bleeding is stopped, so that the invention enables a bleeding site to be located regardless of whether the site is bleeding actively or is clotted. The invention, moreover, enables localization with a high degree of accuracy under a variety of conditions.

U.S. Pat. No. 6,314,314 issued to the inventor of the present invention discloses a technique for localizing an internal bleeding site using a radioactively labeled clotting factor, but the method disclosed in U.S. Pat. No. 6,314,314 is not applicable to MRI, CT, X-ray, PET, or optical imaging techniques.

### SUMMARY OF THE INVENTION

The present invention provides a method for localizing an internal bleeding site whereby a protein or other factor involved in the clotting process is complexed to an imaging agent and injected into a patient believed to be at risk of internal bleeding. A clot in the patient will naturally accumulate a certain concentration of the injected complex, and within a short period of time the concentration becomes sufficient to be detected by an imaging apparatus. The imaging contrast agent may, for example, be an MRI contrast agent, a CT contrast agent, a PET agent, or a fluorescent substance.

### BRIEF DESCRIPTION OF THE DRAWINGS

FIG. 1 is a schematic diagram of an exemplary bleeding site within a human colon;

FIG. 2 is a schematic diagram showing the administration of the diagnostic procedure according to this invention;

FIG. 3 is a flow diagram showing the blood coagulation cascade according to a first known model; and

FIG. 4 is a flow diagram showing the blood coagulation cascade according to a second known model.

### DETAILED DESCRIPTION

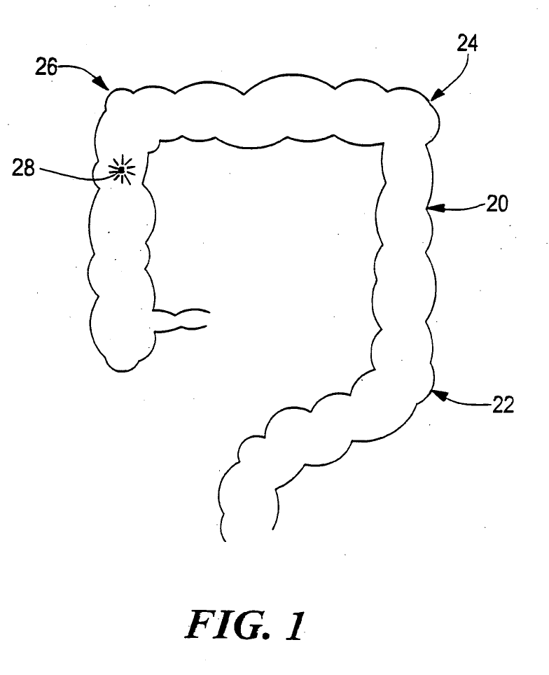

FIG. 1 shows a human colon 20 including a series of characteristic bends 22, 24, and 26 with a bleeding site 28 located past the bend 24. Bleeding in this location would be typical for diverticulosis. Such a bleeding site would be difficult to locate using endoscopy because this site is relatively inaccessible to endocscopes. Similarly, trying to identify such a bleeding site via angiography or a nuclear medicine scan might also fail.

Reference is now made to the coagulation cascade models that have been determined which govern the clotting of a bleeding site such as site 28 of FIG. 1. The most important mechanism that the body employs to stop bleeding is the formation of a clot. Clots are composed of platelets and a number of specialized proteins. At the time of bleeding they collect at the site of a hemorrhage and a clot begins to form. Clotting is a dynamic process that involves initial platelet and protein deposition followed by a continuing deposition process and remodeling.

Bleeding occurs at the site of damage to the layer of cells that line blood vessels. This results in a hole in the vessel that allows egress. The body attempts to repair this hole in the following manner. Platelets and proteins in the blood stream are attracted to the damage by proteins produced by damaged cells and substances in the vessel wall which are exposed by the damage.

These proteins and platelets begin the formation of a plug which will eventually grow to cover and repair the defect. Initially this plug is composed of proteins and platelets. This plug is temporary, and after the bleeding is successfully halted, a complex series of molecular events occurs which greatly increases the strength and durability of the plug and allows the cells underneath the clot to heal and reestablish normal functioning.

Historically, the molecules involved in clotting were divided into two distinct pathways, the intrinsic and extrinsic pathways. Recent work has shown there is considerable overlap between the two pathways and it is more useful to think of the pathways together. The precise details of the clotting cascade are not known, but the major aspects of is clotting are as follows. Damage to a blood vessel exposes tissue factor and other factors that cause platelets to adhere at the site. After a series of molecular events, activated factor VIIa and phospholipid convert factor IX to IXa and X to Xa. These molecules contribute to the production of thrombin (Factor II) from its precursor. Thrombin then converts fibrin to its active form. Fibrin is one of the principal proteins making up the clot. This process of clot formation is a dynamic one, in which weaker areas of the clot may rupture, necessitating repeat of the process in a localized area. In this way, new molecules and platelets are constantly being recruited to the site of bleeding.

At the final stage, activation of Factor XIII helps to cross link fibrin, which stabilizes the clot. Over time the fibrin molecules will link with each other in a dense mesh to form a durable clot. This clot will typically remain for a few days to weeks, depending on the size of the initial hemorrhage.

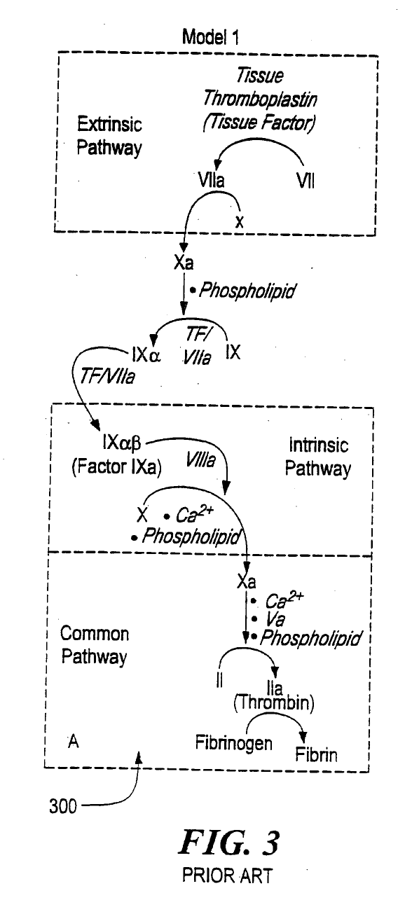

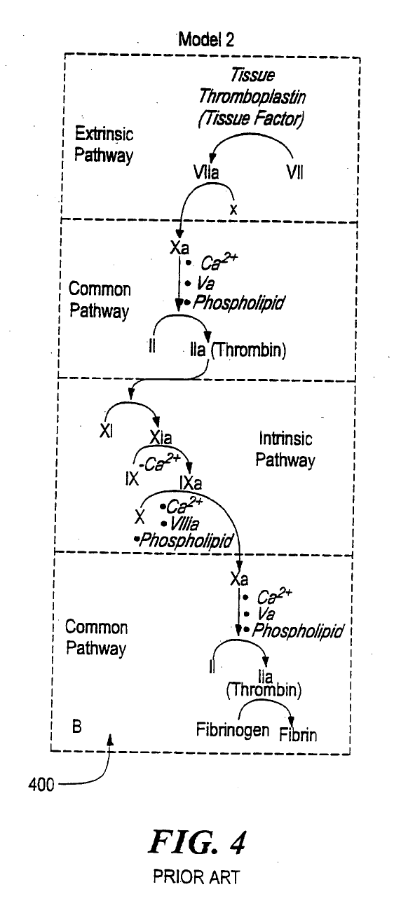

The various factors recruited by the site from the blood stream to enable clotting are shown in FIGS. 3 and 4. The factors in each of the models 300 (FIG. 3) and 400 (FIG. 4) are combined with other elements in the bloodstream such as calcium and phospholipids to eventually form the final clotting products fibrinogen and fibrin. Even if full clotting does not occur, clotting material will be continuously deposited at the bleeding site. And because clotting material is virtually always present, the present invention utilizes the presence of such material as the basis for detecting the exact location of the bleeding site. In this connection, it is noted that, in general, most of the clotting factors will not be present in high concentration in portions of the body other than the actual bleeding site. Thus, by applying an appropriate imaging contrast agent to one or more of the clotting factors shown in the coagulation cascade models 300 and 400 of FIGS. 3 and 4, a product for detecting clotting can be produced.

The following clotting factor or factors, alone and in combination, can be utilized according to this invention: platelets, Factors I, Ia, II, IIa, V, Va, VII, VIIa, VIII, VIIIa, IX, IXa, X, Xa, XI, XIa, XII, XIIa, XIII, XIIIa, fibrinogen, fibrin, fibronectin, von Willebrand's Factor, vinculin, vitronectin, Factor VIIIa and/or b component peptides, ADP, serotonin, platelet factor 4, bethathromboglobulin, high-molecular-weight kininogen, kallikrein, prekallikrein and antithrombin III. Other factors involved in the clotting process can also be used. In general, the factor(s) used should have enough longevity in the clotting process, or should result in by-products that have enough longevity in the clotting process so that they are not reabsorbed into the system too quickly. In addition, the factor(s) used should not be deleterious to health when administered in a detectable concentration and should be present in clots at some time in sufficient concentrations to be detectable. In addition, the factor(s) used should remain sufficiently diluted in other parts of the body so that they do not trick the detector into giving a false reading. In other words, the factor(s) used should not have a substantial affinity for other organs or locations other than the clotting site. Also, the factor(s) used should exhibit sufficient build-up at the clotting site, in a reasonable short period of time, so that they are detectable over and background “noise” generated by remaining freely circulating labeled factors.

The imaging contrast agent may, for example, be one or more of the many known contrast agents used in MRI, CT, PET, fluorescence, or other imaging techniques. Suitable MRI contrast agents include paramagnetic metals such as titanium, vanadium, chromium, manganese, iron, cobalt, nickel, copper, europium, gadolinium and protactinium as disclosed in U.S. Pat. No. 4,615,879 to Runge et al, or a ferromagnetic or superparamagnetic iron oxide such as magnetite and gamma ferric oxide as disclosed in U.S. Pat. No. 4,827,945 to Groman et al. Suitable CT contrast agents include any x-ray opaque material such as iron, calcium, barium, iodine, as routinely used in radiographic imaging. Suitable PET agents include Fluorine 18. Ideal fluorescent substances emit, after suitable excitation, wavelengths to which body tissue and blood are transparent. This allows them to be detected by various imaging equipment. Fluorescent substances that emit in the infrared range have this property, and are therefore ideal imaging agents. Examples include indocyanine green, iodocyanine green, IRDye78, IRDye80, IRDye38, IRDye40, IRDye41, IRDye700, IRDye800, Cy7, IR-786, DRAQ5NO, quantum dots, and analogs thereof. Quantum dots in particular are semiconductor nanocrystals with size-dependent optical and electronic properties such that when illuminated with a primary energy source, they emit at a specific energy frequency. The list of fluorescent substances should not be taken to be complete. Any molecule that can be excited to emit light in the infrared or near-infrared could be similarly complexed to a protein or substance involved in clotting could similarly be used. The entire contents of Runge et al and Groman et al, are incorporated herein by reference.

The method of complexing the contrast agent to the protein is dependent on both the agent and the clotting factor. This is generally done using existing techniques. For example iodination of a protein is a well-known method. The complexed product is provided in an aqueous solution that can include other elements such as saline. More specifically, the complexed product can be provided in concentrations of approximately one milligram per milliliter.

Variants of the current invention include the use of proteins with significant sequence homology to the proteins involved in the clotting process, or formulations employing fragments or portions of the proteins.

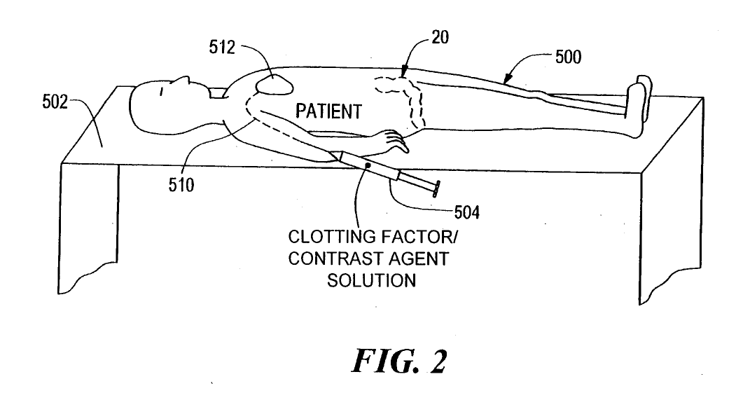

With reference now to FIG. 2, a technique for administering the complexed imaging contrast agent and clotting factor solution of the present invention for locating an internal bleeding site will be explained. The patient 500 is shown reclining on a examination or operating room table 502. Alternatively, the patient can be seated or even standing. A syringe 504 or other device for delivering the solution is applied to the patient's circulatory system shown here as a series of dotted lines 510 interconnected to the heart 512. Typically, a vein in the arm is used. The syringe, an IV bag or some other device carries the solution. The total volume of solution injected can be approximately 10-100 milliliters administered over a time period of approximately a few minutes. The solution enters the bloodstream through the circulatory system 510 and eventually migrates throughout the body until it finds its way to the bleeding site in the colon 20. Clotting is an ongoing process. The approximate time from administration of the solution to the build-up of a sufficiently detectable concentration is approximately a few minutes. At such time, a CT, MRI or PET machine, or a fluoroscope or other appropriate imaging apparatus is utilized to locate the site of the bleeding in the patient by detecting the contrast agent so as to determine a location of the bleeding site based upon a concentration of the contrast agent complexed to the clotting factor.

The foregoing provides a detailed description of a preferred embodiment. Various modifications and additions can be made without departing from the spirit and scope of the invention. In particular, the list of clotting factors set forth herein should not be taken as exhaustive. Additional proteins and other materials involved in various stages of the clotting process can be employed. And similarly, various additional contrast agents other than the contrast agents described herein may be complexed to the clotting factor without departing from the scope of the invention. In addition, the times and dosages described herein can be varied. For example, multiple administrations of different clotting factors and/or different types of clotting factors in each administration can be employed. Accordingly, the foregoing description is meant to be taken only by way of example and not to otherwise limit the scope of the present invention as defined in the appended claims.

What is claimed is:

1. A method for localizing an internal bleeding site in the body of a mammal believed to be at risk of internal bleeding, said method comprising:
introducing into the circulatory system of the mammal a solution comprising a clotting factor that is capable of contributing to clot formation, and a contrast agent complexed to the clotting factor;
permitting time to pass for at least some of the clotting factor in the solution to become localized to the bleeding site to participate in clot formation;
scanning the body of the mammal near a suspected bleeding site with a detector capable of detecting the contrast agent so as to determine a location of the bleeding site based upon a concentration of the contrast agent complexed to the clotting factor.

2. The method according to claim 1, wherein the contrast agent comprises at least one of an MRI contrast agent, a CT contrast agent, a PET agent, and a fluorescent substance.

3. The method according to claim 2, wherein the MRI contrast agent comprises at least one of a paramagnetic contrast agent, a ferromagnetic contrast agent, and a superparamagnetic contrast agent.

4. The method according to claim 3, wherein the MRI contrast agent comprises at least one of: titanium, vanadium, chromium, manganese, iron, cobalt, nickel, copper, europium, gadolinium, protactinium, magnetite and gamma ferric oxide.

5. The method according to claim 2, wherein the CT contrast agent comprises at least one of iron, calcium, barium and iodine.

6. The method according to claim 2, wherein the PET contrast agent comprises Fluorine 18.

7. The method according to claim 2, wherein the fluorescent substance comprises indocyanine green, iodocyanine green, IRDye78, IRDye80, IRDye38, IRDye40, IRDye41, IRDye700, IRDye800, Cy7, IR-786, DRAQ5NO, quantum dots, and analogs thereof.

8. The method according to claim 1, wherein the clotting factor comprises any protein shown to localize to an area of a clot.

9. The method according to claim 2, wherein the clotting factor comprises any protein shown to localize to an area of a clot.

10. The method according to claim 1, wherein the clotting factor comprises at least one of platelets, Factors I, Ia, II, IIa, V, Va, VII, VIIa, VIII, VIIIa, IX, IXa, X, Xa, XI, XIa, XII, XIIa, XIII, XIIIa, fibrinogen, fibrin, fibronectin, von Willebrand's factor, vinculin, vitronectin, Factor VIIIa component peptides, Factor VIIIb component peptides, ADP, serotonin, platelet factor 4, betathromboglobulin, high-molecular weight kininogen, kallikrein, prekallikrein and antithrombin III.

11. The method according to claim 2, wherein the clotting factor comprises at least one of platelets, Factors I, Ia, II, XIIa, V, Va, VII, VIIa, VIII, VIIIa, IX, IXa, X, Xa, XI, XIa, XII, XIIa, XIII, XIIIa, fibrinogen, fibrin, fibronectin, von Willebrand's factor, vinculin, vitronectin, Factor VIIIa component peptides, Factor VIIIb component peptides, ADP, serotonin, platelet factor 4, betathromboglobulin, high-molecular weight kininogen, kallikrein, prekallikrein and antithrombin III.

12. The method as set forth in claim 2 wherein the contrast agent comprises an MRI contrast agent, and the body of the mammal is scanned by placing the mammal in an MRI scanner so as to determine the location of the bleeding site.

13. The method according to claim 2, wherein the contrast agent comprises a CT contrast agent, and the body of the mammal is scanned by placing the mammal in a CT scanner so as to determine the location of the bleeding site.

14. The method as set forth in claim 2 wherein the contrast agent comprises a PET agent, and the body of the mammal is scanned by placing the mammal in a PET scanner so as to determine the location of the bleeding site.

15. The method as set forth in claim 2 wherein the contrast agent comprises a fluorescent substance, and the mammal is scanned by placing a scope into a body cavity suspected of bleeding and exciting and detecting a fluorescent substance so as to determine the location of the bleeding site.

16. The method as set forth in claim 2 wherein the contrast agent comprises a fluorescent substance, and the mammal is scanned while in the operating room with an open body cavity by placing suitable camera over the area of the suspected bleeding site and detecting a fluorescent substance so as to determine the location of the bleeding site.

17. The method according to claim 1, wherein the bleeding site is located in the colon.

18. The method according to claim 1, wherein the bleeding site is located in the small intestine.

19. The method according to claim 1, wherein the bleeding site is located in the stomach.

20. The method according to claim 1, wherein the bleeding site is located in the esophagus.
